(19) United States
(12) Patent Application Publication
(10) Pub. No.: US 2008/0262075 A1
(43) Pub. Date: Oct. 23, 2008

(54) Pyrrolidine Derivatives as Muscarinic Receptor Antagonists

(75) Inventors: Mohammad Salman, Princeton, NJ (US); Pakala Kumara Savithru Sarma, Haryana (IN); Sandeep Y. Shelke, Maharashtra (IN); Anita Chugh, New Delhi (IN); Suman Gupta, Haryana (IN)

Correspondence Address: RANBAXY INC., 600 COLLEGE ROAD EAST, SUITE 2100, PRINCETON, NJ, 08540, US

(73) Assignee: RANBAXY LABORATORIES LIMITED, Haryana (IN)

(21) Appl. No.: 11/573,804

(22) PCT Filed: Aug. 18, 2005
(86) PCT No.: PCT/IB05/02449

§ 371 Date: Feb. 14, 2008

(30) Foreign Application Priority Data
Aug. 19, 2004 (IN) 1550/DEL/2004
Jul. 11, 2005 (IN) 1796/DEL/2005

(51) Int. Cl. A61K 31/40; C07D 207/04; A61P 11/00; A61P 13/00; A61P 1/00

(52) U.S. Cl. 514/428; 548/571

(57) ABSTRACT

This invention relates to pyrrolidine derivatives, which are useful, among other uses, for the treatment of various diseases of the respiratory, urinary and gastrointestinal systems mediated through muscarinic receptors. Processes for the preparation of described compounds, pharmaceutical compositions containing the described compounds and the methods for treating the diseases mediated through muscarinic receptors are also provided.

### FIELD OF THE INVENTION

This invention relates to pyrrolidine derivatives, which are useful, among other uses, for the treatment of various diseases of the respiratory, urinary and gastrointestinal systems mediated through muscarinic receptors. Processes for the preparation of described compounds, pharmaceutical compositions containing the described compounds and the methods for treating the diseases mediated through muscarinic receptors are also provided.

### BACKGROUND OF THE INVENTION

Muscarinic receptors as members of the G Protein Coupled Receptors (GPCRs) are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented. For example, the Ml subtype is located primarily in neuronal tissues such as cereberal cortex and autonomic ganglia, the M₂ subtype is present mainly in the heart where it mediates cholinergically induced bradycardia, and the M₃ subtype is located predominantly on smooth muscle and salivary glands (Nature, 323, p. 411 (1986); Science, 237, p. 527 (1987)).

A review in Current Opinions in Chemical Biology, 3, p. 426 (1999), as well as in Trends in Pharmacological Sciences, 22, p. 409 (2001) by Eglen et. al., describes the biological potentials of modulating muscarinic receptor subtypes by ligands in different disease conditions, such as Alzheimer's Disease, pain, urinary disease condition, chronic obstructive pulmonary disease, and the like.

A review in J. Med. Chem., 43, p. 4333 (2000), by Felder et. al. describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 6, p. 142 (2001).

Birdsall et. al. in Trends in Pharmacological Sciences, 22, p. 215 (2001) have also summarized the recent developments on the role of different muscarinic receptor subtypes using different muscarinic receptor of knock out mice.

Muscarinic agonists such as muscarine and pilocarpine and antagonists such as atropine have been known for over a century, but little progress has been made in the discovery of receptor subtype-selective compounds, making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quaternary derivatives of atropine such as ipratropium bromide are better tolerated than parenterally administered options, but most of these are not ideal anti-cholinergic bronchodilators, due to lack of selectivity for muscarinic receptor sub-types, resulting in dose-limiting side-effects such as thirst, nausea, mydriasis and those associated with the heart such as tachycardia mediated by the M₂ receptor.

Annual Review of Pharmacological Toxicol., 41, p. 691 (2001), describes the pharmacology of the lower urinary tract infections. Although anti-muscarinic agents such as oxybutynin and tolterodine that act non-selectively on muscarinic receptors have been used for many years to treat bladder hyperactivity, the clinical effectiveness of these agents has been limited due to the side effects such as dry mouth, blurred vision and constipation. Tolterodine is considered to be generally better tolerated than oxybutynin. (Steers et. al., in Curr. Opin. Invest. Drugs, 2, 268; Chapple et. al., in Urology, 55, 33; Steers et al., Adult and Pediatric Urology, ed. Gillenwatter et al., pp 1220-1325, St. Louis, Mo.; Mosby. 3^{rd} edition (1996)).

There remains a need for development of new highly selective muscarinic antagonists, which can interact with distinct subtypes, thus avoiding the occurrence of adverse effects.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Patent Nos. 6,174,900, 6,130,232 and 5,948,792; WO 97/45414 are related to 1,4-disubstituted piperidine derivatives; WO 98/05641 describes fluorinated, 1,4-disubstitued piperidine derivatives; WO 93/16018 and WO96/33973 are other references of interest. U.S. Pat. No. 5,397,800 describes 1-azabicyclo[2.2.1]heptanes. U.S. Pat. No. 5,001,160 describes 1-aryl-1-hydroxy-1-substituted-3-(4-substituted-1-piperazinyl)-2-propanones. WO 01/42213 describes 2-biphenyl-4-piperidinyl ureas. WO 01/42212 describes carbamate derivatives. WO 01/90081 describes amino alkyl lactam. WO 02/53564 describes quinuclidine derivatives. WO 02/00652 describes carbamates derived from arylalkyl amines. WO 02/06241 describes 1,2,3,5-tetrahydrobenzo(c)azepin-4-one derivatives.

WO 2004/005252 describes azabicyclo derivatives described as musacrinic receptor antagonists. WO 2004/004629, WO 2004/052857, WO 2004/067510, WO 2004/014853, WO 2004/014363 describes 3,6-disubstituted azabicyclo [3.1.0] hexane derivatives described as useful muscarinic receptor antagonists. WO2004/056811 describes flavoxate derivatives as muscarinic receptor antagonists. WO2004/056810 describes xanthene derivatives as muscarinic receptor antagonists. WO2004/056767 describes 1-substituted-3-pyrrolidine derivatives as muscarinic receptor antagonists. WO2004/089363, WO2004/089898, WO04069835, WO2004/089900 and WO2004089364 describes substituted azabicyclohexane derivatives as muscarinic receptor antagonists. WO 98/00109, 98/00132, 98/00133 and 98/00016 describe isomers of glycopyrolate. U.S. Pat. No. 6,307,060 describes enantiomerically pure basic N-heterocyclicaryl cycloalkyl hydroxy carboxylic esters and their use in medicaments.

U.S. Pat. No. 6,204,285 describes methods and compositions for treating urinary incontinence using enantiomerically enriched (R,R)-glycopyrrolate. WO 03/087094 describes new pyrrolidinium derivatives. A report in J. Med. Chem., 44, p. 984 (2002), describes cyclohexylmethyl piperidinyl triphenylpropioamide derivatives as selective M₃ antagonist discriminating against the other receptor subtypes. Bio-Organic Medicinal Chemistry Letters, 15, p. 2093 (2005) describes synthesis and activity of analogues of Oxybutynin and Tolterodine. Pharmazie, 57(2), 138 (2002) describes glycopyrolate analogues.

### SUMMARY OF THE INVENTION

In one aspect, pyrrolidine derivatives are provided as muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The stereoisomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, stereoisomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I, and their pharmaceutically acceptable salts, pharmaceutically acceptable solvates, esters, stereoisomers, N-oxides, polymorphs, prodrugs or metabolites,

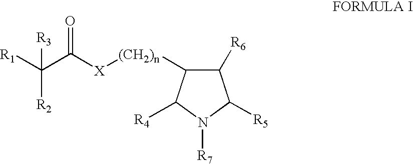

wherein
R₁ and R₂ can be independently selected from alkyl, alkenyl, alkynyl, haloalkyl, cycloalkyl, aryl or heteroaryl.
R₃ can represent hydrogen, lower alkyl, hydroxy, amino or alkoxy.
X can represent oxygen, sulphur or NR₈ (wherein R₈ can represent hydrogen, lower alkyl or aralkyl).
n can represent an integer ranging from 0 to 3.
R₄, R₅ and R₆ can be independently selected from hydrogen or alkyl.
R₇ can represent hydrogen, alkyl, —CHR₉R₁₀ (wherein R₉ and R₁₀ can be independently selected from hydrogen, alkyl or aryl), —(CH₂)ₘ—R₁₁ (wherein R₁₁ is aryl or heteroaryl and m can be an integer from 1 to 3) or —COR₁₂ (wherein R₁₂ represent alkyl, cycloalkyl, aryl, aralkyl or heteroaryl).
with the proviso that R₁, R₂ and R₃ cannot be phenyl, cycloalkyl and hydroxy, respectively, when R₉ and R₁₀ are hydrogen and phenyl, and with the further proviso that when R₇ is (CH₂)ₘ—R₁₁, R₃ is hydrogen.

In accordance with a second aspect, there are provided methods for the treatment or prophylaxis of an animal or human suffering from a disease or disorder of the respiratory, urinary and gastrointestinal systems, wherein the disease or disorder is mediated through muscarinic receptors. The methods include administration of at least one compound having the structure of Formula I.

In accordance with a third aspect, there are provided methods for the treatment or prophylaxis of an animal or human suffering from a disease or disorder associated with muscarinic receptors, comprising administering to a patient in need thereof, an effective amount of a muscarinic receptor antagonist compound as described above.

In accordance with a fourth aspect, there are provided methods for the treatment or prophylaxis of an animal or human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fifth aspect, there are provided processes for preparing the compounds as described above.

The compounds described herein exhibit significant potency in terms of their activity, which was determined by in vitro receptor binding assays. Some compounds were found to function as potent muscarinic receptor antagonists with high affinity towards M₃ receptors. Therefore, pharmaceutical compositions for the possible treatment for the disease or disorders associated with muscarinic receptors are provided. In addition, the compounds can be administered orally or parenterally.

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, C(═O)NR_{f}R_{q}, NHC(═O)NR_{f}R_{q}, —C(═O)heteroaryl, C(═O)heterocyclyl, —O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ-{wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O) R_{f}, C(═O)NR_{f}R_{q}, O—C(═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, C(═O)NR_{f}R_{q}, NHC(═O)NR_{f}R_{q}, —OC(═O)NR_{f}R_{q} (wherein R_{f} and R_{q} are the same as defined earlier), cyano or —SO₂R₆ (where R₆ is same as defined earlier). “Cycloalkylalkyl” refers to alkyl-cycloalkyl group linked through alkyl portion, wherein the alkyl and cycloalkyl are the same as defined earlier.

As used herein the term “alkoxy” refers to the group O-alkyl wherein alkyl is the same as defined above. As used herein the term “haloalkyl” refers to alkyl substituted with halogen. As used herein the term “halogen” refers to fluoro, bromo, chloro or iodo.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

As used herein the term “carboxy” refers to —C(═O)O—R₁₂ wherein R₁₂ is selected from the group consisting of hydrogen, alkyl and cycloalkyl.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z}, or —NHOH}, C(═O)NR_{f}R_{q} and —NHC(═O)NR_{f}R_{q}, —SO₂R₆, —O—C(═O)NR_{f}R_{q}, —O—C(═O)R_{f}, O—C(═O)OR_{f} (wherein R₆, R_{f} and R_{q} are as defined earlier, and R_{z} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term “pharmaceutically acceptable solvates” refers to solvates with waters (i.e hydrates) or pharmaceutically acceptable organic solvents. Such solvates are also encompassed within the scope of this invention.

The phrase “pharmaceutically acceptable salts” of the compounds of Formula I include acid addition salts such as hydrochloride, hydrobromide, hydrofluoric, sulphate, bisulfate, phosphate, hydrogen phosphate, acetate, brosylate, citrate, fumarate, glyconate, lactate, maleate, mesylate, succinate, and tartarate. Quaternary ammonium salts such as alkyl salts, aralkyl salts, and the like, of the organic bases may be readily formed by treatment of the organic bases with the appropriate quaternary salts forming substances, which include, for example methyl chloride, methyl bromide, methyl iodide, methyl sulphate, methyl benzene sulphonate, methyl p-toluene sulphonate, ethyl chloride, ethyl bromide, ethyl iodide, n-propyl chloride, n-propyl bromide, n-propyl iodide, isopropyl bromide, n-butyl chloride, n-butyl bromide, isobutyl bromide, sec-butylbromide, n-amyl bromide, n-hexyl chloride, benzyl chloride, benzyl bromide, and ethyl sulphate.

The present invention also includes within its scope prodrugs of these agents. In general, such “prodrugs” will be functional derivatives of these compounds, which are readily convertible in vivo into the required compound. Conventional procedures for the selection and preparation of suitable prodrug derivatives are described, for example, in “Design of Prodrugs”, ed. H Bundgaard and, Elsevier, 1985.

The present invention also includes metabolites, which become active upon introduction into the biological system.

The crystalline or amorphous forms of compounds described herein may exist as polymorphs and as such are intended to be included in the present invention.

The compounds of present invention include stereoisomers. The term “stereoisomer” refers to compounds, which have identical chemical composition, but differ with regard to arrangement of the atoms and the groups in space. These include, diastereomers, geometrical isomers, atropisomer and conformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformational of a reference compound that converts to the reference compound only slowly on the NMR or laboratory time scale. Conformational isomers (or conformers or rotational isomers or rotamers) are stereoisomers produced by rotation about a bonds, and are often rapidly interconverting at room temperature. Racemic mixtures are also encompassed within the scope of this invention.

### DETAILED DESCRIPTION OF THE INVENTION

The compounds described herein may be prepared by techniques well known in the art and familiar to the average synthetic organic chemist. In addition, the compounds of the present invention may be prepared by the following reaction sequences as depicted in Schemes I, II and III of the accompanying drawings.

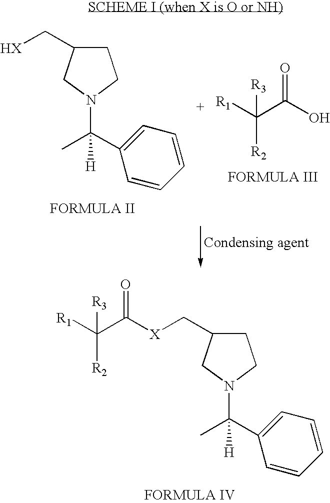

The compounds of Formula IV can be prepared, for example, by the reaction sequence as shown in Scheme I. The preparation comprises coupling a compound of Formula II with a compound of Formula III to give a compound of Formula IV (wherein X, R₁, R₂ and R₃ are the same as defined earlier).

The reaction of a compound of Formula II with a compound of Formula III to give a compound of Formula IV can be carried out in the presence hydroxybenzotriazole and N-methylmorpholine and a coupling agent, for example, 1-(3-dimethylaminopropyl)-3-ethyl carbodiimide hydrochloride (EDC. HCl) or 1,3-dicyclohexylcarbodiimide (DCC). The reaction of a compound of Formula II with a compound of Formula III can be carried out in a solvent, for example, dimethylformamide, chloroform or dimethylsulphoxide.

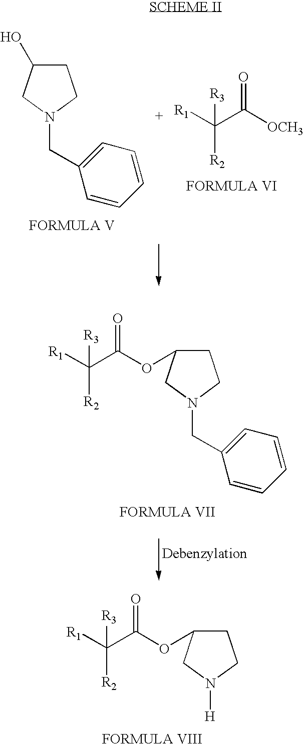

The compounds of Formula VIII can be prepared, for example, by the reaction sequence as shown in Scheme II. The preparation comprises reacting 1-benzyl-pyrrolidin-3-ol of Formula V with a compound of Formula VI to give a compound of Formula VII (wherein R₁, R₂ and R₃ are the same as defined earlier), which on debenzylation gives a compound of Formula VIII.

The reaction of 1-benzyl-pyrrolidin-3-ol of Formula V with a compound of Formula VI to give a compound of Formula VII can be carried out in a solvent, for example, heptane, hexane, toluene or xylene. The reaction of 1-benzyl-pyrrolidin-3-ol of Formula V with a compound of Formula VI can be carried out in the presence of a base, for example, sodium, sodium methoxide or sodium hydride.

The debenzylation of a compound of Formula VII to give a compound of Formula VIII can be carried out in the presence of a debenzylating agent, for example, palladium on carbon and hydrogen or ammonium formate and palladium on carbon. The debenzylation of a compound of Formula VII to give a compound of Formula VIII can be carried out in a solvent, for example, methanol, ethanol or iosproanol, at temperatures ranging from about 50 to about 110° C.

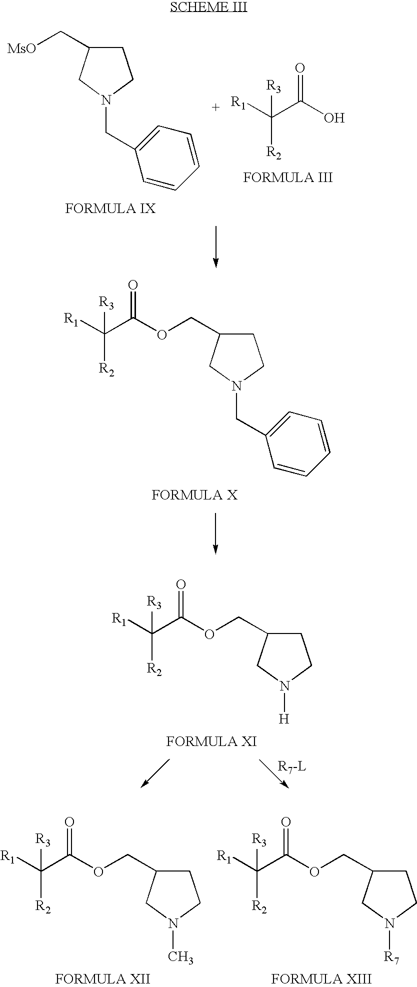

The compounds of Formula XII and XIII can be prepared by the reaction sequence, as shown in scheme III. The preparation comprises reacting 1-benzyl-3-[(methylsulfonyloxy)methyl]-pyrrolidine of Formula IX with a compound of Formula III to give a compound of Formula X (wherein R₁, R₂ and R₃ are the same as defined earlier), which on debenzylation gives a compound of Formula XI, which on reaction with

Path a: formaldehyde gives a compound of Formula XII, or
Path b: a compound of Formula R₇-L (wherein L is any leaving group known in the art, for example, halogen, O-mesyl or O-tosyl group) gives a compound of Formula XIII (wherein R₇ is —(CH₂)ₘ, —R₁₁ wherein R₁₁ and m are the same as defined earlier).

The condensation of a compound of Formula IX with a compound of Formula III to give a compound of Formula X can be carried out in a solvent, for example, benzene, toluene or xylene. The condensation of a compound of Formula IX with a compound of Formula III can be carried out in the presence of a condensing agent, for example, 1,8-diazabicyclo[5.4.0]undecan-7-ene (DBU) or 1,4-diazabicyclo[2.2.2]octane (DABCO).

The debenzylation of a compound of Formula X to give a compound of Formula XI can be carried out in a solvent such as methanol or ethanol. The debenzylation of a compound of Formula X to give a compound of Formula XI can be carried out in the presence of a catalyst such as palladium on carbon and hydrogen gas or ammonium formate and palladium on carbon.

The reaction of a compound of Formula XI with formaldehyde (path a) to give a compound of Formula XII is carried out in the presence of a reducing agent, for example, sodium cyanoborohydride or sodiumtriacetoxyborohydride in a solvent, for example, acetonitrile.

The reaction of a compound of Formula XI with a compound of Formula R₇-L (path b) to give a compound of Formula XIII can be carried out in a solvent, for example, dimethylsulphoxide, acetonitrile or dimethylformamide.

In the above scheme, where specific bases, coupling agents, reducing agents, protecting groups, deprotecting agents, N-alkylating/benzylating agents, solvents, catalysts etc. are mentioned, it is to be understood that other bases, coupling agents, reducing agents, deprotecting agents, N-alkylating/benzylating agents, solvents etc. known to those skilled in art may be used. Similarly, the reaction temperature and duration may be adjusted according to the desired needs.

Particular compounds are shown here (also shown in Table I):
(2R,2S)-[(3′R,3′S)-1′-((R)-α-methyl-benzyl)-pyrrolidin-3′-ylmethyl]-2-hydroxy-2-cyclopentyl-2-phenylacetic acid ester (Compound No. 1),
[(3′R, 3′S)-1′-((R)-α-methyl-benzyl)-pyrrolidin-3′-ylmethyl]-2-hydroxy-2,2-diphenyl acetic acid ester (Compound No. 2),
(2R,2S)-[(3′R, 3′S)-1′-((R)-α-methyl-benzyl)-pyrrolidin-3′-ylmethyl]-2-hydroxy-2-cyclohexyl-2-phenylacetic acid ester (Compound No. 3),
(2R,2S)-N-[(3′R, 3′S)-1′-((R)-α-methyl-benzyl)-pyrrolidin-3′-ylmethyl]-2-hydroxy-2-cyclopentyl-2-phenyl acetamide (Compound No. 4),
(2R,2S)-N-[(3′R, 3′S)-1′-((R)-α-methyl-benzyl)-pyrrolidin-3′-ylmethyl]-2-hydroxy-2-cyclohexyl-2-phenylacetamide (Compound No. 5),
N-[(3′R, 3′S)-1′-((R)-α-methyl-benzyl)-pyrrolidin-3′-ylmethyl]-2-hydroxy-2,2-diphenyl acetamide (Compound No. 6),
(2R,2S)-[(3′R)-1′-((R)-α-methyl-benzyl)-pyrrolidin-3′-ylmethyl]-2-hydroxy-2-cyclopentyl-2-phenyl acetic acid ester (Compound No. 7),
2R-[(3′R)-1′-((R)-α-methyl-benzyl)-pyrrolidin-3′-ylmethyl]-2-hydroxy-2-cyclopentyl-2-phenyl acetic acid ester (Compound No. 8),
2S-[(3′R)-1′-((R)-α-methyl-benzyl)-pyrrolidin-3′-ylmethyl]-2-hydroxy-2-cyclopentyl-2-phenyl acetic acid ester (Compound No. 9),
[(3′R)-1′-((R)-α-methyl-benzyl)-pyrrolidin-3′-ylmethyl]-2-hydroxy-2,2-diphenyl acetic acid ester (Compound No. 10),
2R-[(3′R)-pyrrolidin-3′-yl)-2-hydroxy-2-cyclopentyl-2-phenyl acetic acid ester (Compound No. 11),
(2R,2S)-[((3′R, 3′S)-1′-benzyl-pyrrolidin-3′-ylmethyl)-2-hydroxy-2-(trifluoromethyl)-2-phenyl acetic acid ester (Compound No. 12),
(2R,2S)-[((3′R, 3′S)-pyrrolidin-3′-yl methyl)-2-hydroxy-2-cyclopentyl-2-phenyl acetic acid ester (Compound No. 13),
[((3′R, 3′S)-1′-benzyl-pyrrolidin-3′-yl-methyl)-2-hydroxy-2,2-diphenyl acetic acid ester (Compound No. 14),
(2R,2S)-[((3′R, 3′S)-1′-methyl-pyrrolidin-3′-yl methyl)-2-hydroxy-2-cyclopentyl-2-phenyl acetic acid ester (Compound No. 15),
(2R,2S)-[((3′R, 3′S)-pyrrolidin-3′-yl methyl)-2-hydroxy-2-cyclohexyl-2-phenyl acetic acid ester (Compound No. 16),
(2R,2S)-[((3′R, 3′S)-1′-methyl-pyrrolidin-3′-yl methyl)-2-hydroxy-2-cyclohexyl-2-phenyl acetic acid ester (Compound No. 17),
[((3′R, 3′S)-pyrrolidin-3′-yl methyl)-2-hydroxy-2,2-diphenyl acetic acid ester (Compound No. 18),
[((3′R, 3′S)-1′-methyl-pyrrolidin-3′-yl methyl)-2-hydroxy-2,2-diphenyl acetic acid ester (Compound No. 19),
[((3′R, 3′S)-1′-benzyl-pyrrolidin-3-yl methyl)-2,2-diphenyl acetic acid ester (Compound No. 20),
[((3′R, 3′S)-pyrrolidin-3′-yl methyl)-2,2-diphenyl acetic acid ester (Compound No. 21),
[((3′R, 3′S)-1′-(benzo[1,3]dioxol-5-yl-ethyl)-pyrrolidin-3′-yl methyl)-2,2-diphenyl acetic acid ester (Compound No. 22),
   and their pharmaceutically acceptable salts, pharmaceutically acceptable solvates, esters, stereoisomers, N-oxides, polymorphs, prodrugs, or metabolites.

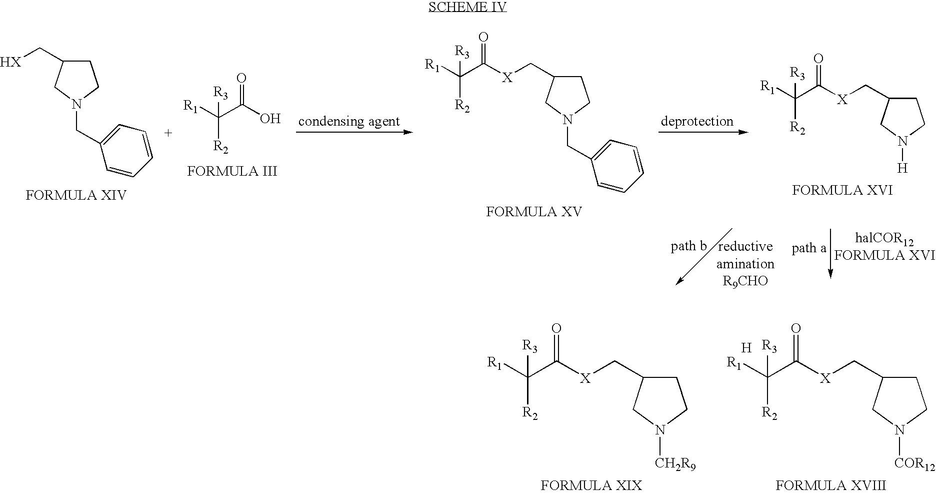

The compounds of Formula XV, XVI, XVIII and XIX can be prepared by following the procedure described in Scheme IV. Thus the preparation comprises condensing a compound of Formula XIV (wherein X is the same as defined earlier) with a compound of Formula III (wherein R₁, R₂ and R₃ are the same as defined earlier) to give a compound of Formula XV, which undergoes deprotection to give a compound of Formula XVI,

Path a: which is reacted with a compound of Formula XVII (wherein hal is Cl, Br or I and R₁₂ is the same as defined earlier) to give a compound of Formula XVIII, or
Path b: which undergoes reductive amination with a compound of Formula R₉CHO (wherein R₉ is the same as defined earlier) to give a compound of Formula XIX.

The condensation of a compound of Formula XIV with a compound of Formula III to give a compound of Formula XV can be carried out in an organic solvent (for example, dimethylformamide, tetrahydrofuran, diethyl ether, chloroform or dioxane) in the presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide).

The deprotection of a compound of Formula XV to give a compound of Formula XVI can be carried out in an organic solvent (for example, methanol, ethanol, propanol or isopropylalcohol) in the presence of a deprotecting agent (for example, palladium on carbon in presence of hydrogen gas or palladium on carbon with a source of hydrogen gas (for example, ammonium formate solution, cyclohexene or formic acid)).

The reaction of a compound of Formula XVI with a compound of Formula XVII (Path a) to give a compound of Formula XVIII can be carried out in an organic solvent (for example, dichloromethane, dichloroethane, carbon tetrachloride or chloroform) in the presence of a base (for example, triethylamine, pyridine, N-methylmorpholine or diisopropylethylamine) and catalyst (for example, dimethylaminopyridine, 4-(pyrrolidino)pyridine.

The reductive amination of a compound of Formula XVI with a compound of Formula R₉CHO to give a compound of Formula XIX (Path b) can be carried out in an organic solvent (for example, selected from, dichloromethane, dichloroethane, chloroform or carbon tetrachloride) with reducing agent (for example, sodium triacetoxyborohydride or sodium cyanoborohydride).

Some illustrative compounds prepared following Scheme IV are:
Hydroxy-diphenyl-acetic acid 1-(2-benzyloxy-acetyl)-pyrrolidin-3-ylmethyl ester (Compound No. 23),
2-Hydroxy-2-phenyl-pent-4-ynoic acid 1-benzyl-pyrrolidin-3-ylmethyl ester (Compound No. 24),
N-(1-Benzyl-pyrrolidin-3-ylmethyl)-2-cyclopentyl-2-hydroxy-N-methyl-2-phenyl-acetamide (Compound No. 25),
2-Cyclopentyl-2-hydroxy-N-methyl-2-phenyl-N-pyrrolidin-3-ylmethyl-acetamide (Compound No. 26),
2-Cyclopentyl-2-hydroxy-N-methyl-N-(1-methyl-pyrrolidin-3-ylmethyl)-2-phenyl-acetamide (Compound No. 27),
N-(1-Benzyl-pyrrolidin-3-ylmethyl)-2-cyclohexyl-2-hydroxy-N-methyl-2-phenyl-acetamide (Compound No. 28),
N-(1-Benzyl-pyrrolidin-3-ylmethyl)-2-hydroxy-N-methyl-2,2-diphenyl-acetamide (Compound No. 29),
2-Cyclohexyl-2-hydroxy-N-methyl-2-phenyl-N-pyrrolidin-3-ylmethyl-acetamide (Compound No. 30),
N-[1-(2-Benzyloxy-acetyl)-pyrrolidin-3-ylmethyl]-2-cyclopentyl-2-hydroxy-N-methyl-2-phenyl-acetamide (Compound No. 31),
   and its pharmaceutically accepted salts, pharmaceutically acceptable solvates, enantiomers, diastereomers, polymorphs or N-oxides.

TABLE I

| | |
|---|---|
| | Formula I |
| 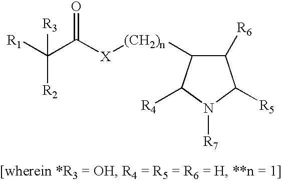 | |

| Comp. | | | | | Configuration | Configuration |
|---|---|---|---|---|---|---|
| No. | R₁ | R₂ | X | R₇ | at C-2 | at pyrrolidine |
| 1 | phenyl | cyclopentyl | O | α-methyl-benzyl | RS | RS |
| 2 | phenyl | Phenyl | O | α-methyl-benzyl | — | RS |
| 3 | phenyl | cyclohexyl | O | α-methyl-benzyl | RS | RS |
| 4 | phenyl | cyclopentyl | NH | α-methyl-benzyl | RS | RS |
| 5 | phenyl | cyclohexyl | NH | α-methyl-benzyl | RS | RS |
| 6 | phenyl | Phenyl | NH | α-methyl-benzyl | — | RS |
| 7 | phenyl | cyclopentyl | O | α-methyl-benzyl | RS | |
| 8 | phenyl | cyclopentyl | O | α-methyl-benzyl | R | R |
| 9 | phenyl | cyclopentyl | O | α-methyl-benzyl | S | R |
| 10 | phenyl | phenyl | O | α-methyl-benzyl | — | R |
| 11 | phenyl | cyclopentyl | O | hydrogen | R | R |
| 12 | phenyl | trifluoromethyl | O | benzyl | RS | RS |
| 13 | phenyl | cyclopentyl | O | hydrogen | RS | RS |
| 14 | phenyl | Phenyl | O | benzyl | — | RS |
| 15 | phenyl | cyclopentyl | O | methyl | RS | RS |
| 16 | phenyl | cyclohexyl | O | Hydrogen | RS | RS |
| 17 | phenyl | cyclohexyl | O | methyl | RS | RS |
| 18 | phenyl | phenyl | O | hydrogen | — | RS |
| 19 | phenyl | phenyl | O | methyl | — | RS |
| 20 | phenyl | phenyl | O | benzyl | — | RS |
| 21 | phenyl | phenyl | O | hydrogen | — | RS |
| 22 | phenyl | phenyl | O | benzo[1,3]dioxol- | — | RS |
| | | | | 5-yl-ethyl | | |
| 23. | phenyl | phenyl | O | Benzyloxy- | — | RS |
| | | | | methylcarbonyl | | |
| 24. | phenyl | propyne | O | benzyl | RS | RS |
| 25. | phenyl | cyclopentyl | —N(CH₃) | benzyl | RS | RS |
| 26. | phenyl | cyclopentyl | —N(CH₃) | hydrogen | RS | RS |
| 27. | phenyl | cyclopentyl | —N(CH₃) | methyl | RS | RS |
| 28. | phenyl | cyclohexyl | —N(CH₃) | benzyl | RS | RS |
| 29. | phenyl | phenyl | —N(CH₃) | benzyl | — | RS |
| 30. | phenyl | cyclohexyl | —N(CH₃) | hydrogen | RS | RS |
| 31. | phenyl | cyclopentyl | —N(CH₃) | benzyloxy- | RS | RS |
| | | | | methylcarbonyl | | |

*R₃ = H for compound No. 20, 21 and 22
**n = 0 for compound No. 11

*R₃=H for compound No. 20, 21 and 22
**n=0 for compound No. 11

Because of their valuable pharmacological properties, the compounds described herein may be administered to an animal for treatment orally, or by a parenteral route. The pharmaceutical compositions described herein can be produced and administered in dosage units; each unit containing a certain amount of at least one compound described herein and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthalmic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

The compounds described herein can be produced and formulated as their stereoisomers, N-Oxides, polymorphs, solvates and pharmaceutically acceptable salts, as well as metabolites having the same type of activity. Pharmaceutical compositions comprising the molecules of Formula I or metabolites, stereoisomers, N-oxides, polymorphs, solvates or pharmaceutically acceptable salts thereof, in combination with pharmaceutically acceptable carrier and optionally included excipient can also be produced.

The examples mentioned below demonstrate general synthetic procedures, as well as specific preparations of particular compounds. The examples are provided to illustrate the details of the invention and should not be constrained to limit the scope of the present invention.

### EXAMPLES

Various solvents, such as acetone, methanol, pyridine, ether, tetrahydrofuran, hexane and dichloromethane were dried using various drying reagents according to the procedures well known in the literature. IR spectra were recorded as nujol mulls or a thin neat film on a Perkin Elmer Paragon instrument, Nuclear Magnetic Resonance (NMR) were recorded on a Varian XL-300 MHz instrument using tetramethylsilane as an internal standard.

### Example A

### Synthesis of (1-benzyl-pyrrolidin-3-ylmethyl)-methyl-amine

Step a: Synthesis of 1-(benzyl-pyrrolidin-3-yl)-methanol

A solution of the compound 1-benzyl-5-oxo-pyrrolidine-3-carboxylic acid methyl ester (1.0 eq.) (commercially available) in toluene was cooled to 0° C. under inert atmosphere. To the mixture was added solution of borane (3.75 eq.) in dimethyl sulphide and refluxed the mixture for 16 hours at 100-110° C. The resulting reaction mixture was cooled to room temperature and subsequently to −5° to −10° C. followed by the addition of sodium bicarbonate solution dropwise. The mixture was slowly brought to room temperature and subsequently refluxed the reaction mixture for 2 hours. The mixture was cooled and organic layer was separated. Aqueous layer was extracted with toluene. The combined toluene layers were washed with water and brine solution. The organic solvent was evaporated under reduced pressure to furnish the title compound. Yield: 99.14%.

Step b: Synthesis of 1-benzyl-3-methanesulphonyl-pyrrolidine

To a solution of the compound obtained from step a above (1.0 eq.) in dichloromethane (10 ml) was added triethylamine (2 eq.) and dimethylaminopyridine (catalytic amount). The mixture was cooled to 0° C. followed by the addition of methane sulphonyl chloride (1.5 eq.) dropwise and stirred the mixture for 14 hours at room temperature. The mixture was diluted with dichloromethane, washed with saturated sodium bicarbonate solution, water and brine, dried over anhydrous sodium sulphate, filtered and concentrated under reduced pressure. The residue thus obtained was purified by column chromatography to furnish the title compound. Yield: 90.53%

Step c: Synthesis of 1-benzyl-pyrrolidin-3-ylmethyl)-methyl-amine

To a solution of the compound obtained from step b above (4.0 g) in methanol (40 ml) was added aqueous methylamine (40%, 40 ml) and heated the mixture for 16 hours at 85-90° C. in autoclave. The solvent was evaporated under reduced pressure and the residue thus obtained was diluted with water and acidified with hydrochloric acid (1:1) and washed with dichloromethane. The aqueous layer was basified with sodium hydroxide solution (20%). The mixture was extracted with ethyl acetate, washed with water and brine, dried over anhydrous sodium sulphate and concentrated under reduced pressure to furnish the title compound. Yield: 84.72%.

### Example 1

### Preparation of (2R,2S)-[(3′R, 3′S)— 1′-((R)-α-methyl-benzyl)-pyrrolidin-3′-ylmethyl]-2-hydroxy-2-cyclopentyl-2-phenylacetic acid ester (Compound No. 1)

To a solution of 2-hydroxy-2-cyclopentyl-2-phenyl acetic acid (prepared following the procedure described in J. Amer. Chem. Soc. 75, 2654 (1953); J. Org. Chem. 2000, 65, 6283-6287) (0.59 g, 2.7 mm) and 1-((R)-α-methyl benzyl)-3-pyrrolidin methanol (0.5 g, 2.4 mm) (prepared according to the method described in J. Med. Chem., 1987, 30, 1711) in dimethylformamide (10.0 ml) at about 0-5° C., hydroxy benzotriazole (0.36 g, 2.7 mm) and N-methylmorpholine (0.54 ml, 4.9 mm) were added and stirred at the same temperature for about 1 hour. 1-(3-dimethylaminopropyl)-3-ethyl carbodiimide hydrochloride (0.48 g, 2.4 mm) was added and stirring was continued for about 1 hour at about 0-5° C. The reaction mixture was stirred at about 25 to 30° C. for about 15 hours. The reaction mixture was poured onto water, extracted with ethyl acetate and ethyl acetate layer was washed with water and brine solution, dried over anhydrous sodium sulphate and concentrated. The residue was purified by silica gel column chromatography using 50% ethyl acetate in hexane to get the title product in 17% (0.17 g) yield.

IR (DCM): 1722.9 cm⁻¹

¹H NMR (CDCl₃): δ 7.59-7.61 (m, 2H), 7.29-7.39 (m, 8H), 4.06-4.07 (m, 2H), 3.68-3.78 (m, 1H), 3.50 (m, 1H), 3.14-3.16 (m, 1H), 2.84 (m, 1H), 2.36-2.56 (m, 4H), 2.10 (s, 1H), 1.70-1.80 (m, 1H), 1.50-1.69 (m, 8H), 1.34-1.47 (m, 3H)

Mass: 408 (M+1)

Similarly the following illustrative compounds were prepared following the procedure described above.

[(3′R, 3′S)-1′-((R)-α-methyl-benzyl)-pyrrolidin-3′-ylmethyl]-2-hydroxy-2,2-diphenyl acetic acid ester (Compound No. 2)

IR (DCM): 1731.8 cm⁻¹

¹H NMR (CDCl₃): δ 7.00-7.45 (m, 15H), 4.16-4.24 (m, 2H), 3.08-3.10 (m, 1H), 2.31-2.50 (m, 4H), 2.02-2.07 (m, 2H), 1.82-1.84 (m, 2H), 1.30-1.33 (m, 3H)

Mass: 416 (M+1)

(2R,2S)-[(3′R, 3′S)-1′-((R)-α-methyl-benzyl)-pyrrolidin-3′-ylmethyl]-2-hydroxy-2-cyclohexyl-2-phenylacetic acid ester (Compound No. 3)

IR (DCM): 1724.0 cm⁻¹

¹H NMR (CDCl₃): δ 7.58-7.60 (m, 2H), 7.30-7.41 (m, 8H), 4.04-4.09 (m, 2H), 3.70 (m, 1H), 3.52 (m, 1H), 3.15-3.50 (m, 2H), 2.37-2.62 (m, 4H), 2.10-2.17 (m, 2H), 1.64-1.83 (m, 3H), 1.08-1.40 (m, 11H)

Mass: 423 (M+1)

(2R,2S)—N-[(3′R, 3′S)-1′-((R)-α-methyl-benzyl)-pyrrolidin-3′-ylmethyl]-2-hydroxy-2-cyclopentyl-2-phenyl acetamide (Compound No. 4)

IR (DCM): 1652.5 cm⁻¹

¹H NMR (CDCl₃): δ 7.56-7.77 (m, 2H), 7.00-7.32 (m, 8H), 3.04-3.21 (m, 4H), 2.56 (m, 1H), 2.22-2.42 (m, 4H), 1.87 (m, 1H), 1.56-1.60 (m, 6H), 1.15-1.30 (m, 8H)

Mass: 408 (M+1)

(2R,2S)—N-[(3′R, 3′S)-1′-((R)-α-methyl-benzyl)-pyrrolidin-3′-ylmethyl]-2-hydroxy-2-cyclohexyl-2-phenylacetamide (Compound No. 5)

IR (DCM): 1654.2 cm⁻¹

Mass: 422 (M+1).

N-[(3′R, 3′S)-1′-((R)-α-methyl-benzyl)-pyrrolidin-3′-ylmethyl]-2-hydroxy-2,2-diphenyl acetamide (Compound No. 6)

IR (DCM): 1658.5 cm⁻¹

Mass: 415 (M+1)

(2R,2S)-[(3′R)-1′-((R)-α-methyl-benzyl)-pyrrolidin-3′-ylmethyl]-2-hydroxy-2-cyclopentyl-2-phenyl acetic acid ester (Compound No. 7)

IR (DCM): 1723.5 cm⁻¹

¹H NMR (CDCl₃): δ 7.59-7.63 (m, 2H), 7.12-7.42 (m, 8H), 4.03-4.08 (m, 2H) 3.68 (m, 1H), 3.49 (s, 3H), 3.16-3.18 (m, 1H), 2.10-2.53 (m, 6H), 1.30-1.73 (m, 9H)

Mass: 407 (M+1)

2R-[(3′R)-1′-((R)-α-methyl-benzyl)-pyrrolidin-3′-ylmethyl]-2-hydroxy-2-cyclopentyl-2-phenyl acetic acid ester (Compound No. 8)

IR (DCM): 1726.2 cm⁻¹

Mass: 408 (M+1)

2S-[(3′R)-1′-((R)-α-methyl-benzyl)-pyrrolidin-3′-ylmethyl]-2-hydroxy-2-cyclopentyl-2-phenyl acetic acid ester (Compound No. 9)

IR (DCM): 1725.1 cm⁻¹

Mass: 408 (M+1)

[(3′R)-1′-((R)-α-methyl-benzyl)-pyrrolidin-3′-ylmethyl]-2-hydroxy-2,2-diphenyl acetic acid ester (Compound No. 10)

IR (DCM): 1729.1 cm⁻¹

¹H NMR (CDCl₃): δ 7.23-7.41 (m, 15H), 4.12-4.20 (m, 2H), 3.06-3.10 (m, 1H), 2.58 (m, 1H), 2.40-2.42 (m, 2H), 2.25-2.27 (m, 1H), 2.04-2.08 (m, 1H), 1.85 (m, 1H), 1.50 (m, 4H)

Mass: 416 (M+1).

### Example 2

### Preparation of 2R-[(3′R)-pyrrolidin-3′-yl)-2-hydroxy-2-cyclopentyl-2-phenyl acetic acid ester (Compound No. 11)

Step a: Preparation of (3R)-1-benzyl-pyrrolidin-3-ol

The compound (3R)-pyrrolidin-3-ol hydrochloride (2.2 g, 17.8 mM) was dissolved in dichloromethane (25.0 ml) and triethylamine (5.0 ml, 35.6 mM) was added at room temperature with constant stirring for about 5 minutes. Benzyl chloride (2.5 ml, 21.4 mM) was added to it in one lot at the same temperature followed by refluxing for about 15 hours. The reaction mixture was diluted with chloroform and 1N sodium hydroxide (15.0 ml) was added with constant stirring for about 10 minutes. The organic layer was separated and washed with aqueous sodium bicarbonate and brine solution. It was further dried over anhydrous sodium sulphate and concentrated to get the title compound.

Step b: Preparation of 2R-[((3′R)-1′-benzyl-pyrrolidin-3′-yl)]-2-hydroxy-2-cyclopentyl-2-phenyl acetic acid ester

A mixture of methyl (2R)-2-cyclopentyl-2-hydroxy-2-phenyl acetic acid ester (4.5 g, 19.2 mm) and (3R)-1-benzyl-pyrrolidin-3-ol (3.5 g, 19.8 mm) in heptane (600.0 ml) was refluxed under a Dean and Stark apparatus with the addition of piece of sodium (20 mg cover) at 0° C. After about 5 hours refluxing, methanol (3.0 ml) was added at room temperature followed by water (50.0 ml). The organic layer was separated and aqueous layer was extracted with n-heptane. The combined organic layer was washed with water and brine solution. Dried, evaporated and the residue was purified by silica gel column chromatography using ethyl acetate in hexane to afford the product in 62% (4.5 g) yield.

IR (DCM): 1703.8 cm⁻¹

¹H NMR (CDCl₃): δ 7.64-7.66 (m, 2H), 7.28-7.35 (m, 8H), 5.21-5.23 (m, 1H), 3.49-3.75 (m, 3H), 2.70-2.91 (m, 3H), 2.46-2.52 (m, 2H), 2.24-2.29 (m, 1H), 1.90 (m, 1H), 1.31-1.66 (m, 8H).

Mass: 380 (M+1).

Step c: Preparation of 2R-[((3′R)-1′-pyrrolidin-3′-yl)]-2-hydroxy-2-cyclopentyl-2-phenyl acetic acid ester

To a solution of 2R-[((3′R)-1′-benzyl-pyrrolidin-3′-yl)]-2-hydroxy-2-cyclopentyl-2-phenyl acetic acid ester (1.3 g, 30.5 mmole) in dry methanol (25.0 mL), 5% palladium on carbon (0.2 g), (50% wet) was added under nitrogen. Then anhydrous ammonium formate (0.8 g, 12.38 mmole) was added under stirring and the reaction mixture was refluxed for half an hour under nitrogen atmosphere. Cooled to room temperature and the reaction mixture was filtered through a bed of hyflo. The hyflo bed was washed with methanol (75.0 mL), ethyl acetate (25.0 mL) and water (25.0 mL). The filterate was concentrated under vacuum. The residue was diluted with water and pH of the resulting solution was adjusted to (pH˜14) with 1N sodium hydroxide. Extracted with ethyl acetate (2×50 mL) and the ethyl acetate layer was washed with water and brine solution. Dried over anhydrous sodium sulphate and concentrated to give the title compound.

IR (DCM): 1727.1 cm⁻¹

¹H NMR (CDCl₃): δ 7.62-7.64 (m, 2H), 7.24-7.36 (m, 3H), 5.32 (m, 1H), 2.89-3.17 (m, 6H), 2.09 (m, 1H), 1.90 (m, 1H), 1.29-1.65 (m, 9H)

Mass: 290 (M+1).

### Example 4

### Preparation of (2R,2S)-[((3′R, 3′S)-1′-benzyl-pyrrolidin-3′-ylmethyl)-2-hydroxy-2-(trifluoromethyl)-2-phenyl acetic acid ester (Compound No. 12)

To a solution of 2-trifluoromethyl-2-hydroxy-2-phenylacetic acid (prepared following the procedure described in J. Amer. Chem. Soc. 75, 2654 (1953); J. Org. Chem. 2000, 65, 6283-6287) (1.9 g, 8.33 mmol) and (3R,3S)-1-benzyl-3-[(methylsulfonyloxy)methyl]-pyrrolidine (prepared following the procedure described in J. Med. Chem., 1987, 30, 1711) (2.4 g, 8.5 mmol) in toluene (20 ml), 1,8-diazabicyclo[5.4.0]undecan-7-ene (DBU) (1.6 g, 8.33 mmol) was added and the mixture was refluxed for overnight. It was quenched by addition of aqueous sodium bicarbonate solution. The organic layer was separated and washed with water, brine and dried over anhydrous sodium sulphate. The organic layer was filtered and evaporated to give crude product. The crude product was purified by silica gel column chromatography.

IR (DCM): 1747.2 cm⁻¹

¹H NMR (CDCl₃): δ 7.74-7.76 (m, 2H), 7.31-7.39 (m, 8H), 4.23-4.35 (m, 2H), 3.54-3.67 (m, 2H), 2.41-2.67 (m, 5H), 1.98-2.04 (m, 1H), 1.53-1.56 (m, 1H), 1.28 (m, 1H).

Mass: 394 (M+1)

Similarly, the following illustrative compounds were prepared following the procedure described above

[((3′R, 3′S)-1′-benzyl-pyrrolidin-3′-yl-methyl)-2-hydroxy-2,2-diphenyl acetic acid ester (Compound No. 14)

IR (DCM): 1731.1 cm⁻¹

¹H NMR (CDCl₃): δ 7.26-7.41 (m, 15H), 4.14-4.20 (m, 2H0, 3.51 (s, 2H), 2.37-2.53 (m, 5H), 2.09-2.13 (m, 1H), 1.83-1.89 (m, 2H)

Mass: 402 (M+1).

[((3′R, 3′S)-1′-benzyl-pyrrolidin-3-yl methyl)-2,2-diphenyl acetic acid ester (Compound No. 20)

IR (DCM): 1735.2 cm⁻¹

¹H NMR (CDCl₃): δ 7.24-7.33 (m, 15H), 5.00 (s, 1H), 4.03-4.13 (m, 2H), 3.54 (s, 2H), 2.44-2.64 (m, 4H), 2.10-2.19 (m, 2H), 1.42-1.44 (m, 1H).

Mass: 386 (M+1).

### Example 5

### Preparation of (2R,2S)-[((3′R, 3′S)-pyrrolidin-3′-yl methyl)-2-hydroxy-2-cyclopentyl-2-phenyl acetic acid ester (Compound No. 13)

To a solution of ((3′R)-1′-benzyl-pyrrolidin-3′-yl methyl)-2-hydroxy-2-cyclopentyl-2-phenyl acetic acid ester (prepared as in example 2) (1.3 g, 30.5 mmole) in dry methanol (25.0 mL), 5% palladium on carbon (0.2 g), (50% wet) was added under nitrogen. Then anhydrous ammonium formate (0.8 g, 12.38 mmole) was added under stirring and the reaction mixture was refluxed for half an hour under nitrogen atmosphere. Cooled to room temperature and the reaction mixture was filtered through a bed of hyflo. The hyflo bed was washed with methanol (75.0 mL), ethyl acetate (25.0 mL) and water (25.0 mL). The filterate was concentrated under vacuum. The residue was diluted with water and pH of the resulting solution was adjusted to (pH˜14) with 1N sodium hydroxide. Extracted with ethyl acetate (2×50 mL) and the ethyl acetate layer was washed with water and brine solution. Dried over anhydrous sodium sulphate and concentrated to give the title compound.

IR (DCM): 1726.6 cm⁻¹

Mass: 304 (M+1)

Similarly, the following illustrative compounds were prepared following the procedure described above

(2R,2S)-[((3′R, 3′S)-pyrrolidin-3′-yl methyl)-2-hydroxy-2-cyclohexyl-2-phenyl acetic acid ester (Compound No. 16)

IR (DCM): 1727.2 cm⁻¹

¹H NMR (CDCl₃): δ 7.61-7.64 (m, 2H), 7.24-7.36 (m, 3H), 4.07-4.15 (m, 2H), 2.91-3.01 (m, 3H), 2.57-2.62 (m, 5H), 2.44 (m, 1H), 1.83 (m, 2H), 1.65-1.66 (m, 2H), 1.13-1.45 (m, 7H).

Mass: 318 (M+1), 300 (M—OH)

[((3′R, 3′S)-pyrrolidin-3′-yl methyl)-2-hydroxy-2,2-diphenyl acetic acid ester (Compound No. 18)

IR (DCM): 1734.0 cm⁻¹

¹H NMR (CDCl₃): δ 7.25-7.40 (m, 10H), 4.16-4.22 (m, 2H), 2.81-2.91 (m, 2H), 2.53-2.59 (m, 1H), 2.40-2.42 (m, 1H), 1.81-1.83 (m, 1H), 1.28-1.37 (m, 3H)

Mass: 312 (M+1)

[((3′R, 3′S)-pyrrolidin-3′-yl methyl)-2,2-diphenyl acetic acid ester (Compound No. 21)

IR (DCM): 1737.8 cm⁻¹

Mass: 296 (M+1)

### Example 6

### Preparation of (2R 2S)-[((3′R, 3′S)-1′-methyl-pyrrolidin-3′-ylmethyl)-2-hydroxy-2-cyclopentyl-2-phenyl acetic acid ester (Compound No. 15)

To a solution of (2R,2S)-[((3′R, 3′S)-(1-pyrrolidin-3′-ylmethyl)]-2-hydroxy-2-cyclopentyl-2-phenyl acetic acid ester (prepared in example-5) (0.3 g, 0.99 mm) in acetonitrile (18.0 ml), formaldehyde (37.1 ml, 2.5 mm) and sodium cyanoborohydride (0.23 g) were added at room temperature and stirred for about 1 hour. Acetic acid (0.5 ml) was added to the reaction mixture and stirring continued for 2 more hours at room temperature. Acetonitrile was evaporated and the residue was diluted with water (50.0 ml) and basified with aqueous sodium hydroxide. Extracted with ethyl acetate (6×500 ml) and the ethyl acetate layer was washed with water and brine solution dried, evaporated and the residue was purified by silica gel column chromatography using 10% methanol in dichloromethane to get product in 60% yield.

IR (DCM): 1729.6 cm⁻¹

¹H NMR (CDCl₃): δ 7.62-7.65 (m, 2H), 7.28-7.36 (m, 3H), 4.07-4.1H (m, 2H), 2.91-2.94 (m, 1H), 2.51-2.62 (m, 4H), 2.35 (s, 3H), 2.26 (m, 11H), 1.90-2.00 (m, 1H), 1.28-1.62 (m, 10H)

Mass: 318 (M+1)

Similarly, the following illustrative compounds were prepared following the procedure described above

(2R,2S)-[((3′R, 3′S)-1′-methyl-pyrrolidin-3′-yl methyl)-2-hydroxy-2-cyclohexyl-2-phenyl acetic acid ester (Compound No. 17)

IR (DCM): 1728.5 cm⁻¹

¹H NMR (CDCl₃): δ 7.61-7.63 (m, 2H), 7.23-7.35 (m, 3H), 4.06-4.14 (m, 2H), 2.52-2.59 (m, 4H), 2.33-2.34 (d, J=3 Hz, 3H), 2.22-2.25 (m, 2H), 2.01 (m, 1H), 1.80 (m, 1H), 1.63-1.65 (m, 2H), 1.11-1.46 (m, 9H)

Mass: 332 (M+1)

[((3′R, 3′S)-1′-methyl-pyrrolidin-3′-yl methyl)-2-hydroxy-2,2-diphenyl acetic acid ester (Compound No. 19)

IR (DCM): 1737.4 cm⁻¹

¹H NMR (CDCl₃): δ 7.35-7.41 (m, 10H), 4.29-4.31 (m, 2H), 4.09 (m, 1H), 2.60-2.80 (m, 2H), 2.51-2.53 (m, 3H), 2.45-2.46 (m, 1H), 2.00-2.01 (m, 1H), 1.61 (m, 2H)

Mass: 326 (M+1)

### Example 7

### Preparation of [((3′R,3′S)-1′-(benzo[113]dioxol-5-yl-ethyl)-pyrrolidin-3′-yl methyl)-2,2-diphenyl acetic acid ester (Compound No. 22)

The compound [((3′R, 3′S)-(1′-pyrrolidin-3′-yl methyl)-2,2-diphenyl acetic acid ester was dissolved in acetonitrile and 5-(2-bromoethyl-1,3-benzodioxole was added. To the reaction mixture, potassium carbonate and potassium iodide were added. The reaction mixture was heated under refluxed for about 9 hours. The reaction mixture was cooled to room temperature and acetonitrile was evaporated under vacuum. The residue was partitioned between ethyl acetate and water. The organic layer was washed with water and brine solution followed by drying over anhydrous sodium sulphate and then concentrated. The residue was purified by silica gel column chromatography using 20% methanol in chloroform to get the title compound.

IR (DCM): 1734.7 cm⁻¹

¹H NMR (CDCl₃): δ 7.27-7.30 (m, 3H), 6.96 (s, 1H), 6.58-6.85 (m, 6H), 5.92-5.95 (m, 4H), 5.57 (d, J=18 Hz, 1H), 5.12 (d, J=12 Hz, 1H), 3.27-3.32 (m, 2H), 3.06-3.11 (m, 2H), 2.80-2.82 (m, 1H), 1.55 (s, 3H), 1.28-1.42 (m, 5H)

### Example 8

### Synthesis of N-(1-benzyl-pyrrolidin-3-ylmethyl)-2-cyclopentyl-2-hydroxy-N-methyl-2-phenyl-acetamide (Compound No. 25)

To a solution of the compound 2-cyclopentyl-2-hydroxy-2-phenyl acetic acid (1 eq.) in dimethylformamide was added hydroxybenzotriazole (1.5 eq.), 1-(3-dimethylaminopropyl)-3-ethylcarbodiimide hydrochloride (1 eq.) and dimethylaminopyridine (catalytic amount). The reaction mixture was stirred at 15-20° C. for 2 hours followed by the addition of N-methylmorpholine (2 eq.) and a solution of the compound (1-benzyl-pyrrolidin-3-ylmethyl)-methyl-amine (1 eq.) in dimethylformamide (10 ml). The resulting reaction mixture was stirred at 15-20° C. for 1 hour and subsequently at room temperature for 14 hours. To the mixture was added water and stirred for 15 minutes. The aqueous layer was extracted with ethylacetate. The organic layer was washed with sodium bicarbonate solution, water and brine, dried over anhydrous sodium sulphate and concentrated under reduced pressure. The residue thus obtained was purified by column chromatography to furnish the title compound. Yield: 75.34%.

Mass (m/z): 407.0 (M⁺+1).

IR: 1623.8 cm⁻¹, 2951.2 cm⁻¹, 3357.5 cm⁻¹.

¹H NMR: 1.256-1.663 (m, 10H), 2.014-2.559 (m, 6H), 2.713-2.951 (m, 4H), 3.399-3.461 (m, 2H), 3.801 (s, 2H), 7.105-7.334 (m, 10H).

The following illustrative compounds were prepared similarily by coupling an appropriate acid with an appropriate amine or alcohol,

2-Hydroxy-2-phenyl-pent-4-ynoic acid 1-benzyl-pyrrolidin-3-ylmethyl ester (Compound No. 24)

Mass (m/z): 364.0 (M⁺+1)

IR: 1745.2 cm⁻¹, 2923.7 cm⁻¹, 3412.7 cm⁻¹

¹H NMR: 1.86-1.96 (m, 4H), 2.03-2.59 (m, 6H), 2.79-2.91 (m, 3H), 4.04-4.09 (m, 2H), 7.31-7.74 (m, 10H).

N-(1-Benzyl-pyrrolidin-3-ylmethyl)-2-cyclohexyl-2-hydroxy-N-methyl-2-phenyl-acetamide (Compound No. 28)

Mass (m/z): 421.0 (M⁺+1)

IR: 1623.0 cm⁻¹, 2925.3 cm⁻¹, 3383.8 cm⁻¹

¹H NMR (CDCl₃): 1.17-1.37 (m, 6H), 1.41-1.48 (m, 2H), 1.72-2.61 (m, 12H), 2.75-3.62 (m, 6H), 7.20-7.39 (m, 10H).

N-(1-Benzyl-pyrrolidin-3-ylmethyl)-2-hydroxy-N-methyl-2,2-diphenyl-acetamide (Compound No. 29)

Mass (m/z): 415.0 (M⁺+1)

IR: 1747.5 cm⁻¹, 2925.1 cm⁻¹, 3421.4 cm⁻¹

¹H NMR (CDCl₃): 1.20-2.01 (m, 3H), 2.12-2.43 (m, 4H), 2.87-2.91 (m, 2H), 2.95-3.27 (m, 4H), 3.77 (s, 2H), 7.01-7.32 (m, 15H).

### Example 9

### Synthesis of 2-cyclopentyl-2-hydroxy-N-methyl-2-phenyl-N-pyrrolidin-3-ylmethyl-acetamide (Compound No. 26

To a solution of the Compound No. 25 (1 eq.) in methanol (20 times) was added palladium on carbon (10% dry) and ammonium formate. The mixture was refluxed for 25 minutes. The mixture was cooled and filtered through celite bed. The bed was washed with dichloromethane and basified the aqueous layer with sodium hydroxide (2N) to a pH 14. The mixture was extracted with ethylacetate. The organic layer was washed with water and brine, dried over anhydrous sodium sulphate, filtered and concentrated under reduced pressure to furnish the title compound. Yield: 96.36%

Mass (m/z): 317.0 (M⁺+1).

IR: 1625.7 cm⁻¹, 2958.0 cm⁻¹, 3375.7 cm⁻¹

¹H NMR: 1.25-1.32 (m, 2H), 1.60-1.72 (m, 6H), 1.95-2.05 (m, 5H), 2.77-3.04 (m, 8H), 3.17-3.48 (m, 2H), 7.28-7.42 (m, 5H).

The following illustrative compounds were prepared similarily.

2-Cyclohexyl-2-hydroxy-N-methyl-2-phenyl-N-pyrrolidin-3-ylmethyl-acetamide (Compound No. 30)

Mass (m/z): 331.0 (M⁺+1)

IR: 1620.0 cm⁻¹, 2926.7 cm⁻¹, 3385.4%

¹H NMR (CDCl₃): 1.10-1.32 (m, 10H), 1.66-1.70 (m, 4H), 1.98-2.04 (m, 4H), 2.40-2.87 (m, 5H), 3.35 (s, 2H).

### Example 10

### Synthesis of 2-cyclopentyl-2-hydroxy-N-methyl-N-(1-methyl-pyrrolidin-3-ylmethyl)-2-phenyl-acetamide (Compound No. 27)

To a solution of the Compound No. 4 (0.2 g) in acetonitrile (10-15 ml) was added 37% aqueous formaldehyde (1.7 ml) and sodium triacetoxyborohydride (0.16 g) and stirred at room temperature for one hour. Added acetic acid till the pH of reaction mixture is neutral. The reaction mixture was stirred for 2 hours at room temperature. Evaporated acetonitrile completely and added water. Basified to pH 14 with aqueous sodium hydroxide solution (10%). Extracted with ethyl acetate. The organic layer was separated, washed with water and brine, dried over anhydrous sodium sulphate, filtered and concentrated under reduced pressure to furnish the title compound. Yield: 52.67%.

Mass (m/z): 331.0 (M⁺+1)

IR: 1623.6 cm⁻¹, 2925.6 cm⁻¹, 3383.7 cm⁻¹

¹H NMR: 1.256-1.334 (m, 4H), 1.450-1.513 (m, 7H), 1.587-1.764 (m, 8H), 2.449-2.754 (m, 2H), 2.970-3.409 (m, 4H), 7.30-7.415 (m, 5H).

### Example 11

### Synthesis of N-1-(2-benzyloxy-acetyl)-pyrrolidin-3-ylmethyl]-2-cyclopentyl-2-hydroxy-N-methyl-2-phenyl-acetamide (Compound No. 31)

To a solution of the Compound No. 26 (1 eq.) in dichloromethane (10 ml) was added triethylamine (2 eq.) and dimethylaminopyridine (catalytic amount). The mixture was cooled to 0-5° C. and added benzyloxy acetyl chloride (1.5 eq.). The reaction mixture was stirred at 0-5° C. for 30 minutes followed by stirring at room temperature for 16 hours. The mixture was quenched with saturated sodium bicarbonate solution. The organic layer was separated and aqueous layer was extracted with dichloromethane. The combined organic layers were washed with water and brine, dried over anhydrous sodium sulphate and concentrated under reduced pressure. The residue thus obtained was purified by column chromatography to furnish the title compound. Yield: 88.53%

Mass (m/z): 465 (M⁺+1), 487 (M⁺+Na).

IR: 1630 cm⁻¹, 2927.1 cm⁻¹, 3391.9 cm⁻¹.

¹H NMR: 1.12-1.26 (m, 4H), 1.61-1.75 (m, 6H), 2.32-2.37 (m, 6H), 2.88-3.38 (m, 4H), 4.13-4.63 (m, 6H), 7.23-7.41 (m, 10H).

The following illustrative compound was prepared similarily.

### Hydroxy-diphenyl-acetic acid 1-(2-benzyloxy-acetyl)-pyrrolidin-3-ylmethyl ester (Compound No. 23)

Mass (m/z): 460.0 (M⁺+1).

IR: 1707.9 cm⁻¹, 2926.8 cm⁻¹, 3031.6 cm⁻¹

¹H NMR: 1.255 (s, 2H), 3.30-3.88 (m, 2H), 3.966-4.250 (m, 4H), 4.485-4.87 (m, 5H), 5.183-5.285 (m, 1H), 7.225-7.406 (m, 15H).

### Biological Activity

### Radioligand Binding Assays:

The affinity of test compounds for M₂ and M₃ muscarinic receptor subtypes were determined by [³H]-N-Methylscopolamine (NMS) binding studies using rat heart and submandibular gland respectively as described by Moriya et al., (Life Sci, 1999, 64(25): 2351-2358) with minor modifications. Specific binding of [³H]-NMS was also determined using membranes from Chinese hamster ovary (CHO) cells expressing cloned human muscarinic receptor subtypes.

Membrane Preparation:

(a) Rat tissues

Submandibular glands and heart were isolated and placed in ice-cold homogenising buffer (HEPES 20 mM, 10 mM EDTA, pH 7.4) immediately after sacrifice. The tissues were homogenised in ten volumes of homogenising buffer and the homogenate was filtered through two layers of wet gauze and filtrate was centrifuged at 500 g for 10 min. The supernatant was subsequently centrifuged at 40,000 g for 20 min. The pellet thus obtained was resuspended in homogenising buffer (HEPES 20 mM, EDTA 10 mM, pH 7.4) and were stored at −70° C. until the time of assay.

(b) CHO cells expressing human recombinant receptors

The cell pellets were homogenised for 30 sec at 12,000 to 14,000 rpm, with intermittent gaps of 10-15 sec in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer containing 10% sucrose and was stored at −70° C. until the time of assay.

### Ligand Binding Assay:

The compounds were dissolved and diluted in dimethyl sulphoxide. The membrane homogenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fiber filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μl of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki was calculated from competitive binding studies by using Cheng & Prusoff's equation (Biochem Pharmacol, 1973, 22: 3099-3108), Ki=IC₅₀/(1+[L]/Kd), where [L] is the concentration of ligand [³H]-N-methyl scopolamine used in the particular experiment and Kd is the estimate of affinity of receptors to the ligand. The final result is expressed as the pKi value—the negative logarithm of Ki.

Compound Nos. 1-31 exhibited Ki in the range of about 1000 nM to about 0.4 nM at rat M₂ muscarinic receptors, for example, from about 40 nM to about 0.4 nM, or from about 6 nM to about 0.4 nM. Compound Nos. 1-31 exhibited Ki in the range of about 1000 nM to about 0.1 nM at rat M₃ muscarinic receptors, for example from about 65 nM to about 0.1 nM, or from about 10 nM to about 0.1 nM.

While the present invention has been described in terms of its specific embodiments, certain modifications and equivalents will be apparent to those skilled in the art and are intended to be included within the scope of the present invention.

1. A compund having the structure of Formula I, 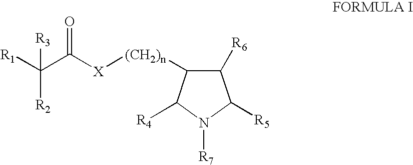
and their pharmaceutically acceptable salts, pharmaceutically acceptable solvates, esters, stereoisomers, N-oxides, polymorphs, prodrugs, or metabolites, wherein
R₁ and R₂ can be independently selected from alkyl, alkenyl, alkynyl, haloalkyl, cycloalkyl, aryl or heteroaryl
R₃ can represent hydrogen, lower alkyl, hydroxy, amino or alkoxy.
X can represent oxygen, sulphur or NR₈ (wherein R₈ can represent hydrogen, lower alkyl or aralkyl),
n can represent an integer ranging from 0 to 3.
R₄, R₅ and R₆ can be independently selected from hydrogen or alkyl.
R₇ can represent hydrogen, alkyl, —COR₁₂ (wherein R₁₂ represent alkyl, cycloalkyl, aryl, aralkyl or heteroaryl), —CHR₉R₁₀ (wherein R₉ and R₁₀ can be independently selected from hydrogen, alkyl or aryl) or —(CH₂)ₘ—R₁₁ (wherein R₁₁ is aryl or heteroaryl and in can be an integer from 1 to 3),
with the proviso that R₁, R₂ and R₃ cannot be phenyl, cycloalkyl and hydroxy, respectively, when R₉ and R₁₀ are hydrogen and phenyl, and with the further proviso that when R₇ is (CH₂)ₘ—R₁₁, R₃ is hydrogen.

2. A compound according to claim 1 wherein R₁ is aryl.

3. (canceled)

4. A compound according to claim 1 wherein R₂ is aryl, cycloalkyl, haloalkyl or alkynyl.

5. (canceled)

6. A compound according to claim 1 wherein R₃ is hydrogen or hydroxy.

7. A compound according to claim 1 wherein X is oxygen, —N(CH₃) or —NH.

8. A compound according to claim 1 wherein n is 0 or 1.

9. A compound according to claim 1 wherein R₄, R₅ and R₆ are hydrogen.

10. A compound according to claim 1 wherein 1<7 is hydrogen or alkyl.

11. (canceled)

12. A compound according to claim 1 wherein R₇ is —CHR₉R₁₀ wherein R₉ and R₁₀ are independently hydrogen, alkyl or aryl.

13. (canceled)

14. A compound according to claim 1 wherein R₇ is —(CH₂)ₘ—R₁₁ wherein R₁₁ is heteroaryl and m is 2.

15. A compound according to claim 1 wherein R₇ is (CH₂)ₘ—R₁₁ wherein R₁₁ is benzo[1.3]dioxol-5-yl-ethyl and m is 2.

16. A compound according to claim 1 wherein R₇ is —COR₁₂.

17. A compound according to claim 1 wherein R₁₂ is optionally substituted alkyl.

18. A compound which is:
(2R, 2S)-[(3′R, 3′S)-1′-((R)-α-methyl-benzyl)-pyrrolidin-3′-ylmethyl]-2-hydroxy-2-cyclopentyl-2-phenylacetic acid ester (Compound No. 1),
[(3′R, 3′S)-1′-((R)-α-methyl-benzyl)-pyrrolidin-3′-ylmethyl]-2-hydroxy-2,2-diphenyl acetic acid ester (Compound No. 2),
(2R,2S)-[(3′R, 3′S)-1′-((R)-α-methyl-benzyl)-pyrrolidin-3′-ylmethyl]-2-hydroxy-2-cyclohexyl-2-phenylacetic acid ester (Compound No. 3),
(2R,2S)-N-[(3′R, 3′S)-1′-((R)-α-methyl-benzyl)-pyrrolidin-3′-ylmethyl]-2-hydroxy-2-cyclopentyl-2-phenyl acetamide (Compound No. 4),
(2R,2S)-N-[(3′R, 3′S)-1′-((R)-α-methyl-benzyl)pyrrolidin-3′-ylmethyl]-2-hydroxy-2-cyclohexyl-2-phenylacetamide (Compound No. 5),
N-[(3′R,3′S)-1′-((R)-α-methyl-benzyl)-pyrolidin-3′-ylmethyl]-2-hydroxy-2,2-diphenyl acetamide (Compound No. 6),
(2R,2S)-[(3′R)-1′-((R)-α-methyl-benzyl)-pyrrolidin-3′-ylmethyl]-2-hydroxy-2-cyclopentyl-2-phenyl acetic acid ester (Compound No. 7),
2R-[(3′R)-1′-((R)-α-methyl-benzyl)-pyrrolidin-3′-ylmethyl]-2-hydroxy-2-cyclopentyl-2-phenyl acetic acid ester (Compound. No. 8),
2S-[(3′R)-1′-((R)-α-methyl-benzyl)-pyrolidin-3′-ylmethyl]-2-hydroxy-2-cyclopentyl-2-phenyl acetic acid ester (Compound No. 9),
[(3′R)-1′-((R)-α-methyl-benzyl)-pyrolidin-3′-ylmethyl]-2-hydroxy-2,2-diphenyl acetic acid ester (Compound No. 10),
2R-[(3′R)-pyrrolidin-3′-yl)-2-hydroxy-2-cyclopentyl-2-phenyl acetic acid ester (Compound No. 11),
(2R,2S)-[((3′R, 3′S)-1′-benzyl-pyrrolidin-3′-ylmethyl)-2-hydroxy-2-(trifluoromethyl)-2-phenyl acetic acid ester (Compound No. 12),
(2R,2S)-[((3′R, 3′S)-pyrrolidin-3′-yl methyl)-2-hydroxy-2-cyclopentyl-2-phenyl acetic acid ester (Compound No. 13),
[((3′R,3′S)-1′-benzyl-pyrrolidin-3′-yl-methyl)-2-hydroxy-2,2-diphenyl acetic acid ester (Compound No. 14),
(2R, 2S)-[((3′R, 3′S)-1′-methyl-pyrrolidin-3′-yl methyl)-2-hydroxy-2-cyclopentyl-2-phenyl acetic acid ester (Compound No. 15),
(2R,2S)-[((3′R,3′S)-pyrrolidin-3′-yl methyl)-2-hydroxy-2-cyclohexyl-2-phenyl acetic acid ester (Compound No. 16),
(2R, 2S)-[((3′R, 3′S)-1′-methyl-pyrrolidin-3′-yl methyl)-2-hydroxy-2-cyclohexyl-2-phenyl acetic acid ester (Compound No. 17),
[((3′R,3′S)-pyrrolidin-3′-yl methyl)-2-hydroxy-2,2-diphenyl acetic acid ester (Compound No. 18),
[((3′R,3′S)-1′-methyl-pyrrolidin-3′-yl methyl)-2-hydroxy-2,2-diphenyl acetic acid ester (Compound No. 19),
[((3′R,3′S))-1′-benzyl-pyrrolidin-3-yl methyl)-2,2-diphenyl acetic acid ester (Compound No. 20),
[((3′R,3′S)-pyrrolidin-3′-yl methyl)-2,2-diphenyl acetic acid ester (Compound No. 21),
[((3′R, 3′S)-1′-(benzo[1,3]dioxo-5-yl-ethyl)-pyrrolidin-3′-yl methyl)-2,2-diphenyl acetic acid ester (Compound No. 22),
Hydroxy-diphenyl-acetic acid 1-(2-benzyloxy-acetyl)-pyrrolidin-3-ylmethyl ester (Compound No. 23),
2-Hydroxy-2-phenyl-pent-4-ynoic acid 1-benzyl-pyrrolidin-3-ylmethyl ester (Compound No. 24),
N-(1-Benzyl-pyrrolidin-3-ylmethyl)-2-cyclopentyl-2-hydroxy-N-methyl-2-phenyl-acetamide (Compound No. 25),
2-Cyclopentyl-2-hydroxy-N-methyl-2-phenyl-pyrrolidin-3-ylmethyl-acetamide (Compound No. 26),
2-Cyclopentyl-2-hydroxy-N-methyl-N-(1-methyl-pyrrolidin-3-ylmethyl)-2-phenyl-acetamide (Compound No. 27),
N-(1-Benzyl-pyrrolidin-3-ylmethyl)-2-cyclohexyl-2-hydroxy-N-methyl-2-phenyl-acetamide (Compound No, 28),
N-(1-Benzyl-pyrrolidin-3-methyl)-2-hydroxy-N-methyl-2,2-diphenyl-acetamide (Compound No. 29),
2-Cyclohexyl-2-hydroxy-N-methyl-2-phenyl-N-pyrrolidin-3-ylmethyl-acetamide (Compound No. 30),
N-[1-(2-Benzyloxy-acetyl)-pyrrolidin-3-ylmethyl]-2-cyclopentyl-2-hydroxy-N-methyl-2-phenyl-acetamide (Compound No. 31),
and their pharmaceutically acceptable salts, pharmaceutically acceptable solvates, esters, stereoisomers, N-oxides, polymorphs, prodrugs, or metabolites,

19. A pharmaceutical composition comprising a therapeutically effective amount of a compound as defined in claim 1-18 optionally together with pharmaceutically acceptable carriers, excipients or diluents.

20. A method for treatment or prophylaxis of an animal or a human suffering from a disease or disorder of the respiratory, urinary and gastrointestinal systems, wherein the disease or disorder is mediated through muscarinic receptors, comprising administering to said animal or human, a therapeutically effective amount of a compound according to claim 18.

21. The method according to claim 20 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis

22. The method for treatment or prophylaxis of an animal or a human suffering from a disease or disorder of the respiratory, urinary and gastrointestinal systems, where the disease or disorder is mediated though muscarinic receptors, comprising administering to said animal or human, a therapeutically effective amount of the pharmaceutical composition according to the claim 19.

23. The method according to claim 22 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes and gastrointestinal hyperkinesis.

24. A process of preparing a compound of Formula IV and its pharmaceutically acceptable salts, pharmaceutically acceptable solvates, esters, stereoisomers, N-oxides, polymorphs, prodrugs or metabolites, wherein the reaction comprises of following steps: 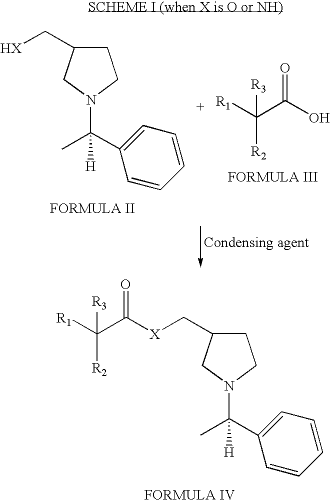
R₁, R₂, R₃ and X are the same as defined in claim 1.

25. (canceled)

26. A process of preparing a compound of Formula VIII, and its pharmaceutically acceptable salts, pharmaceutically acceptable solvates, esters, stereoisomers, N-oxides, polymorphs, prodrugs or metabolites, wherein the reaction comprises of following steps: 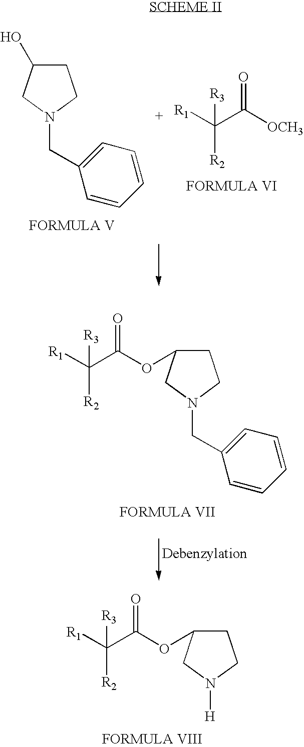
R₁, R₂, R₃ and X are the same as defined in claim 1.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)
